(19) United States
(12) Patent Application Publication
(10) Pub. No.: US 2012/0310135 A1
(43) Pub. Date: Dec. 6, 2012

(54) BLOOD TREATMENT DEVICE AND METHOD FOR ADJUSTING OPERATING PARAMETERS OF A BLOOD TREATMENT DEVICE

(76) Inventors: René Bauer, Mid-levels (HK); Alfred Gagel, Litzendorf (DE); Stefan Kellermann, Massbach (DE); Thomas Nuernberger, Burkardroth (DE); Jochen Schneider, Wipfeld (DE)

(21) Appl. No.: 13/482,621

(22) Filed: May 29, 2012

Related U.S. Application Data
(60) Provisional application No. 61/491,428, filed on May 31, 2011.

(30) Foreign Application Priority Data
May 31, 2011 (DE) 10 2011 102 872.6

(51) Int. Cl. A61M 1/14

(52) U.S. Cl. 604/6.11

(57) ABSTRACT

The present invention relates to a blood treatment device, in particular a dialysis machine, with at least one adjusting means by means of which the blood pump delivery rate can be adjusted, with detection means for detecting at least two physical attributes, wherein the same each are a physical attribute of a patient to be treated with the blood treatment device, and wherein the blood treatment device is formed such that by means of the adjusting means the blood pump delivery rate can be adjusted and/or limited with reference to the detected at least two physical attributes. Furthermore, the present invention relates to a method for adjusting operating parameters of a blood treatment device.

The present invention relates to a blood treatment device and to a method for adjusting operating parameters of a blood treatment device.

In all kinds of hemodialysis the blood of a patient suffering from renal insufficiency is cleaned in an extracorporeal blood circuit. The blood often is delivered by an occluding peristaltic hose pump, but other pump systems, in particular impeller pumps (centrifugal pumps) are also possible.

At least parts, in particular the blood-delivering pump, of the extracorporeal blood circuit can be combined in a cassette system as disposable.

The blood flow rate is an important parameter here and can be adjusted within wide limits in the machine controller by operator input. For example, blood flow rates between 30 ml/min and 600 ml/min are possible. The correct blood flow depends for example on the type of treatment (hemodialysis, hemodiafiltration, hemofiltration, single-needle treatment, etc.), but also on the patient himself, who tolerates the individually maximum blood flow rates in terms of his health.

In young patients particular caution is required, since their circulation can react quite sensitively to too high blood flow rates of the extracorporeal blood circuit. In the pediatric treatment, a certain maximum infused air quantity per unit time must not be exceeded. In the dialysis treatment it can occur that for example due to leakages in the vacuum range of the extracorporeal circuit air is sucked in from the environment and air bubbles are caused in the extracorporeal blood circuit, whose volume typically lies between about 1 μl and 10 μl. The total air volume per unit time, which is infused into the patient in the case of leakages in the vacuum range of the extracorporeal blood circuit, depends on the blood flow rate. The higher the blood flow rate, the higher the infused total volume per unit time in the case of leakages in the vacuum range of the extracorporeal blood circuit. In order to by no means exceed the admissible maximum quantity of infused air per unit time, the blood pump delivery rate has so far been limited to 230 ml/min for young patients, independent of which physical attributes the patient had.

230 ml/min is the blood pump delivery rate which safely supplies not more than the maximum possible air quantity per minute to the patient with a body weight below 10 kg. Young patients, however, very well can have a body weight of more than 10 kg. If a patient for example has a body weight of 30 kg, the limitation of the blood pump rate to 230 ml/min leads to an unnecessarily long dialysis treatment which therefore is encumbering to the patient.

From US 2011/0017667 A1 a limitation of the flow rates in dependence on the body weight is already known, which here however is directed only to patients with a body weight of less than 20 kg.

Therefore, it is the object of the present invention to develop a blood treatment device and a method for adjusting operating parameters of a blood treatment device as mentioned above in an advantageous way, in particular to the effect that in young patients a safe treatment becomes possible, which at the same time is hardly encumbering, as it is effected with an adapted treatment time.

In accordance with the invention, this object is solved by a blood treatment device with the features of claim 1. Accordingly, it is provided that a blood treatment device comprising at least one adjusting means, by means of which the blood pump delivery rate can be adjusted, is provided with a detection means for detecting at least two physical attributes, wherein the physical attributes each constitute a physical attribute of a patient to be treated with the blood treatment device and wherein the blood treatment device is formed such that by means of the adjusting means the blood pump delivery rate can be adjusted and/or limited with reference to the detected at least two physical attributes.

For example, the blood treatment machine can be a dialysis machine.

By employing the at least two physical attributes, a corresponding adaptation of the blood pump delivery rate also can be effected in young patients, even if the same have a body weight of more than 20 kg. In contrast to the pre-definition practiced so far, a patient-individual adjustment of the blood pump delivery rate now is possible. At the same time, however, it is also ensured that in particular the maximum admissible infused air quantity per minute is not exceeded. It thereby is ensured on the one hand that in particular the young patient can safely be treated, but on the other hand the treatment need not be performed longer than necessary.

Advantageous aspects of the invention can be taken from the sub-claims following the main claim.

It can be provided that a first and/or a second physical attribute is the body surface area, the body weight, the body height, the used vessel access and/or the blood viscosity. In particular, for the amount of limitation of the blood pump delivery rate not only the weight of the patient alone, but for example also the body surface area of the patient is relevant. A calculation of the body surface area can be effected for example according to the calculation methods explained below. By means of the advantageously integrated means for detecting the physical attributes, such as the body height and the body weight, the blood treatment device is equipped to determine for example the body surface area by means of these measured data and correspondingly automatically limit the delivery rate of the blood pump. With such configuration, the operator advantageously need not enter any body data, which increases the convenience and prevents the risk of input errors. Furthermore, the blood pump delivery rate can also be limited by other parameters. In particular, the blood viscosity can be employed. The blood viscosity primarily is not directly related to the infused air quantity, but the thicker the patient blood, the less good its flow through the extracorporeal blood circuit. The components of the extracorporeal blood circuit likewise can have a limiting effect on the delivery rate of the blood pump. For example, a simple catheter access only provides for smaller blood delivery rates as compared to a fistula access or the access via an artificial vessel piece.

In particular, it can be provided that the first and second physical attributes are body surface area and vessel access. From the information and combination of these two attributes body surface area and vessel access the blood pump delivery rate expediently can be adjusted in a particularly advantageous way, i.e. can be determined and limited with regard to safety on the one hand and with regard to the best possible duration of treatment on the other hand. Due to the type of vessel access the influence of this attribute might be taken into account when determining the blood pump delivery rate, and taking account of the body surface area of the patient allows to by no means exceed the maximum admissible quantity of infused air per unit time.

Furthermore, it can be provided that the first, second and a third physical attribute are body surface area, body weight and height or body weight, height and vessel access, or body weight, height and blood viscosity. By taking account of the combination of three physical attributes, an even more accurate adjustment and/or limitation of the blood pump delivery rate becomes possible. A first group of physical attributes can be body surface area, body weight and height of the patient. A further group of physical attributes can be body weight, height and vessel access. A further group of physical attributes also can be body weight, height and blood viscosity. In principle, it is also imaginable to evaluate all physical attributes mentioned so far in combination and relation. As already explained above, the attributes vessel access and blood viscosity allow to include specified fluid-dynamic marginal conditions in the determination and limitation of the blood pump delivery rate. The remaining physical attributes such as body surface area, body weight, height of the patient, are employed in particular to by no means exceed the maximum admissible quantity of infused air per unit time, but at the same time determine the maximum possible blood pump delivery rate, in order to save the patient from an unnecessarily long dialysis treatment which therefore is encumbering for the patient.

It is particularly advantageous when the determination of the physical attributes and/or the adjustment and/or limitation of the blood pump delivery rate is effected automatically by means of the blood treatment device. This involves the advantage that the operator need not enter any body data, which increases the convenience and prevents the risk of input errors.

It can also be provided that at least the first or at least the second detection means is a detection means for detecting the body height, wherein the detection means for detecting the body height preferably is and/or comprises a camera, a gauge stick with corresponding sensors such as at least one potentiometer, at least one strain gauge, at least one optical sensor and/or at least one magnetic sensor.

Furthermore, it is conceivable that at least the first or at least the second detection means comprises a connecting means by means of which the blood treatment device preferably can be connected with a detection means and wherein by means of this detection means a physical attribute of a patient to be treated with the blood treatment device can be detected. Thus, it is conceivable that other devices are provided, which interact with the blood treatment device. For example, a patient supporting device with corresponding means such as a scale, sensors for height determination, for example pressure sensors integrated in the chair, can be provided, in order to be able to determine body height, body weight and directly and/or indirectly the body surface area of the patient.

Furthermore, it can be provided that at least the first or at least the second detection means is a detection means by means of which the blood viscosity can be detected. For this purpose, for example the pressure values measured by means of the arterial pressure sensor can be employed. These values provide a clue to the viscosity of the blood, so that the viscosity of the blood can indirectly be calculated. For example in a case in which the arterial pressure is too low, the blood pump delivery rate thus can additionally be limited beyond the extent previously determined from the body weight and the body height. It is, however, also conceivable to determine the viscosity of the blood with reference to the torque of a centrifugal pump which serves as blood pump.

In addition, it can be provided that at least the first or at least the second detection means is a detection means by means of which the vessel access of the patient can be detected. It is conceivable to enter the type of vessel access via an operator input or by inserting a patient card, on which the individual vessel access is stored, into the blood treatment device and thereby include the same in the adjustment and/or limitation of the blood pump delivery rate. The operator input concerning the vessel access also can already consist in that the patient ID is entered in the blood treatment device. In this connection it is furthermore conceivable that the data concerning the vessel access are already stored in the blood treatment device in a memory means and due to the input of the patient ID a corresponding allocation can be effected by means and on the part of the blood treatment device.

It is likewise conceivable that data which directly or indirectly determine the blood pump rate, such as the body weight, the body height, the body surface area and/or the vessel access, are stored in a database to which the blood treatment device has access—for example via a network. Such database for example can be provided in a central computer.

Furthermore, the present invention relates to a method for adjusting operating parameters of a blood treatment device with the features of claim 10. Accordingly, it is provided that in a method for adjusting operating parameters of a blood treatment device the blood pump delivery rate is adjusted and/or limited as at least one operating parameter, wherein at least one first and at least one second physical attribute of a patient to be treated by means of the blood treatment device is detected and wherein in dependence on the detected physical attributes the blood pump delivery rate is adjusted and/or limited.

In addition, it can be provided that the first and/or the second physical attribute is the body surface area, the body weight, the body height, the used vessel access and/or the blood viscosity.

In addition, it is possible that the first and second physical attributes are body surface area and vessel access.

Furthermore, it is conceivable that the first, second and a third physical attribute are body surface area, body weight and height or body weight, height and vessel access, or body weight, height and blood viscosity.

It can also be provided that the determination of the physical attributes and/or the adjustment and/or limitation of the blood pump delivery rate is effected automatically.

It is particularly advantageous when the method is performed with a blood treatment device according to any of claims 1 to 9.

Further details and advantages of the invention will now be explained with reference to an exemplary embodiment of the invention described below.

In a blood treatment device according to the invention, which is configured as dialysis machine, integrated means for determining the body height and the body weight accordingly are provided. As a result, the dialysis machine is able to determine the body surface area of the patient to be treated by means of these measured data and correspondingly automatically limit the delivery rate of the blood pump. Configured in this way, the operator need not enter any body data, which increases the operating comfort of the dialysis machine. At the same time, the risk of input errors is prevented. As means for determining the body height cameras, gauge sticks with corresponding sensors such as potentiometers, strain gauges, optical sensors, magnetic sensors etc. or other devices can be provided, which interact with the dialysis machine. Preferably, it is also provided that the patient supporting device is equipped with corresponding means, such as a scale, sensors for height determination, for example pressure sensors integrated in the chair.

At the same time, the blood treatment device is able to limit the blood pump delivery rate also with reference to other parameters. The blood viscosity primarily is not directly related to the infused air quantity. It should be noted, however, that the thicker the patient blood the less good its flow in the extracorporeal blood circuit of the blood treatment device, whereby in the final analysis the maximum blood pump delivery rate and hence the possibly infused air quantity is influenced indirectly. The arterial pressure sensor provides a clue to the viscosity of the blood. If the arterial pressure is too low, the blood pump delivery rate of the dialysis machine correspondingly is limited in addition beyond the extent previously determined from the body weight and the body height. Alternatively or in addition it is also conceivable to determine the viscosity of the blood by means of the torque of the centrifugal pump, which can serve as blood pump of the dialysis machine, and include this quantity in the determination and limitation of the blood pump delivery rate. In the dialysis machine of the invention, the type of vessel access of the extracorporeal blood circuit at the same time is optionally included in the determination and limitation of the blood pump delivery rate. This is based on the fact that the type of vessel access of the extracorporeal blood circuit also has a limiting effect on the delivery rate of the blood pump. For example, a simple catheter access only provides for smaller blood delivery rates as compared to a fistula access or the access via an artificial vessel piece. The type of vessel access also can be communicated to the machine by operator input or patient card.

In particular, this is based on the knowledge that for the amount of limitation of the delivery rate of the blood pump of the dialysis machine not only the weight of the patient alone is decisive, but for example also the body surface area of the patient. The body surface area can be calculated for example from the calculation formula

$$Body surface area ( {m}^{2} ) = \sqrt{\frac{height ( cm ) * weight ( kg )}{3600}}$$

(Source: Mosteller R D. Simplified Calculation of body surface area. NEJM 1987; 317:1098.).

With reference to the body surface area, the weight and the height of the patient the maximum blood pump rate to be adjusted then can already be determined by means of these parameters alone, but in principle also by employing further parameters. An adaptation then can be effected in that the previously maximum possible blood pump delivery rate of 230 ml/min for patients with a body weight of 20 kg correspondingly is adjusted upwards or downwards. For example, for a body weight of 20 kg to <30 kg a maximum blood pump delivery rate of 310 ml/min is obtained, for 30 kg to <40 kg a blood pump delivery rate of 470 ml/min, and for a body weight of ≧40 kg a blood pump delivery rate of 600 ml/min.

Furthermore, it is conceivable to perform the indication of a weight class by the dialysis machine in dependence on the weight determined or entered. It is conceivable to consider three weight classes. There can be provided a first weight class in the range from 10 kg-20 kg, a second weight class in the range from >20 kg-30 kg, and a third weight class of >30 kg.

1. A blood treatment device, in particular a dialysis machine, comprising at least one adjusting means by means of which a blood pump delivery rate can be adjusted, at least one detection means for detecting at least two physical attributes, wherein the physical attributes each are physical attributes of a patient to be treated with the blood treatment device and wherein the blood treatment device is formed such that by means of the adjusting means the blood pump delivery rate can be adjusted and/or limited with reference to the detected at least two physical attributes.

2. The blood treatment device according to claim 1, characterized in that a first, second and a third physical attribute are body surface area, body weight and height or body weight, height and vessel access, or body weight, height and blood viscosity.

3. The blood treatment device according to claim 1, characterized in that one of the physical attributes is the body surface area, the body weight, the body height, the used vessel access and/or the blood viscosity.

4. The blood treatment device according to claim 1, characterized in that the determination of the physical attributes and/or the adjustment and/or limitation of the blood pump delivery rate is effected automatically by means of the blood treatment device.

5. The blood treatment device according to claim 1, characterized in that one of the at least two detection means is a detection means for detecting the body height, wherein the detection means for detecting the body height preferably is and/or comprises a camera, a gauge stick with corresponding sensors such as at least one potentiometer, at least one strain gauge, at least one optical sensor and/or at least one magnetic sensor.

6. The blood treatment device according to claim 1, characterized in that one of the at least two detection means is a detection means for detecting the body weight, wherein the detection means for detecting the body weight preferably is a pressure sensor.

7. The blood treatment device according to claim 1, characterized in that one of the at least two detection means is a detection means by means of which the blood viscosity can be detected.

8. The blood treatment device according to claim 1, characterized in that one of the at least two detection means is a detection means by means of which the vessel access of the patient can be detected.

9. The blood treatment device according to claim 1, characterized in that one of the at least two detection means comprises a connecting means by means of which the blood treatment device can be connected with a detection means and wherein by means of this detection means a physical attribute of a patient to be treated with the blood treatment device can be detected.

10. A method for adjusting operating parameters of a blood treatment device, wherein the blood pump delivery rate is adjusted and/or limited as at least one operating parameter, wherein at least two physical attributes of a patient to be treated by means of the blood treatment device are detected and wherein in dependence on the detected physical attributes the blood pump delivery rate is adjusted and/or limited.

11. The method according to claim 10, characterized in that a first, second and a third physical attribute are body surface area, body weight and height or body weight, height and vessel access, or body weight, height and blood viscosity.

12. The method according to claim 10, characterized in that one of the at least two physical attributes each is the body surface area, the body weight, the body height, the used vessel access and/or the blood viscosity.

13. The method according to claim 10, characterized in that the determination of the physical attributes and/or the adjustment and/or limitation of the blood pump delivery rate is effected automatically.

14. A method for adjusting operating parameters of a blood treatment device, wherein the blood pump delivery rate is adjusted and/or limited as at least one operating parameter, wherein at least two physical attributes of a patient to be treated by means of the blood treatment device are detected, wherein in dependence on the detected physical attributes the blood pump delivery rate is adjusted and/or limited, and wherein the method is performed with a blood treatment device according to claim 1.
